(19) United States
(12) Patent Application Publication
(10) Pub. No.: US 2026/0184665 A1
(43) Pub. Date: Jul. 2, 2026

(54) METHOD OF SYNTHESIZING ACYL ESTER USING MICROREACTOR

(71) Applicant: INDUSTRIAL TECHNOLOGY RESEARCH INSTITUTE, Hsinchu (TW)

(72) Inventors: Chao-Wu LIAW, Taoyuan City (TW); Chih-Lung CHIN, Hsinchu City (TW); Wan-Chi CHEN, Hsinchu City (TW); Wei WANG, Hsinchu City (TW)

(73) Assignee: INDUSTRIAL TECHNOLOGY RESEARCH INSTITUTE, Hsinchu (TW)

(21) Appl. No.: 19/205,560

(22) Filed: May 12, 2025

(30) Foreign Application Priority Data
Dec. 31, 2024 (TW) 113151680

(51) Int. Cl. C07C 67/40

(52) U.S. Cl. CPC C07C 67/40

(57) ABSTRACT

A method of synthesizing acyl ester using a microreactor, including: injecting an acyl chloride, an alcohol, an organic solvent, an organic base and a calcium oxide into a microreactor by a mixed jet flow, wherein the acyl chloride is injected into the microreactor through a first channel; the calcium oxide is injected into the microreactor through a second channel; wherein each of the alcohol, the organic base and the organic solvent is independently injected into the microreactor through the second channel after being mixed with the calcium oxide, or is independently injected into the microreactor through the first channel after being mixed with the acyl chloride, and the injecting velocity ratio of the first channel to the second channel is 6 to 60.

### CROSS REFERENCE TO RELATED APPLICATIONS

The present application is based on, and claims priority from, Taiwan Application Serial Number 113151680, filed on Dec. 31, 2024, the disclosure of which is hereby incorporated by reference herein in its entirety.

### TECHNICAL FIELD

The technical field relates to a method of synthesizing acyl ester using a microreactor.

### BACKGROUND

Ester compounds are widely found in natural products and synthetic materials, and their characteristics include good volatility, low toxicity, and high chemical tunability, which render them important in the fields of food, pharmaceutical, cosmetic, and industry. Among them, acyl ester monomers are widely used in the fields of semiconductors, panels, and industry because they possess high reactivity, excellent optical transparency, weather resistance and abrasion resistance, as well as good adhesion and processability.

The production of acrylate esters is generally achieved through an esterification reaction involving acyl chlorides and alcohols, typically performed in batch processes. This reaction is notably exothermic and results in complex by-products that are difficult to purify. To prevent abrupt temperature rises that could trigger uncontrolled reactions, traditional batch methods require cooling systems to keep the temperature below 0° C. Additionally, in large-scale industrial operations, solid organic salts often form during the reaction and exhibit poor flowability. These solids can accumulate and clog pipelines, leading to increased system pressure. Such blockages may damage reactors or cause pump failures, which significantly compromise the safety and stability of the process. As a result, maintaining steady operation becomes increasingly challenging.

### SUMMARY

In one embodiment of the disclosure, the method of synthesizing acyl ester using a microreactor includes injecting an acyl chloride, an alcohol, an organic solvent, an organic base, and a calcium oxide into the microreactor by a mixed jet flow. The acyl chloride is injected into the microreactor through a first channel of the microreactor. The calcium oxide is injected into the microreactor through a second channel of the microreactor. Each of the alcohol, the organic base and the organic solvent is independently injected into the microreactor through the second channel of the microreactor after being mixed with the calcium oxide, or is injected into the microreactor through the first channel of the microreactor after being mixed with the acyl chloride. A jet flow velocity ratio of the first channel to the second channel is 6 to 60.

### BRIEF DESCRIPTION OF THE DRAWINGS

The disclosure will become fully understood from the detailed description given hereinbelow and the accompanying drawings which are given by way of illustration only and thus are not limitative of the disclosure, and wherein:

FIG. 1 is a flowchart of the method of synthesizing acyl ester using a microreactor according to one embodiment of the disclosure.

FIG. 2 is a schematic diagram of the method of synthesizing acyl ester using the microreactor according to one embodiment of the disclosure.

FIG. 3 is a particle size diagram of solid organic salt byproducts generated during the synthesis of acyl esters using the microreactor according to one embodiment of the disclosure.

FIG. 4 is a particle size diagram of the solid organic salt byproducts generated during the synthesis of acyl esters using the microreactor according to a comparative example of the disclosure.

FIG. 5 is a particle size diagram of the solid organic salt byproducts generated during the synthesis of acyl esters using the microreactor according to another comparative example of the disclosure.

### DETAILED DESCRIPTION

Disclosed embodiments provide a method of synthesizing acyl ester using a microchannel reactor. In particular, the inventors have found that according to the embodiments, by adding calcium oxide during acyl ester synthesis helps minimize the formation of bulky solid organic salt byproducts, which may prevent blockages in the pipelines. It also reduces the production of hydrochloric acid addition byproducts, thereby improving the overall yield. This method allows the reaction to take place at room temperature, which helps lower energy consumption. Additionally, performing the reaction in a microchannel reactor facilitates continuous production, which increases reaction efficiency. After the reaction is complete, the target acyl ester product can be easily obtained through filtration, simplifying the process and reducing wastewater generation. A detailed description is given in the following discussion of disclosed embodiments.

In the following detailed description, for purposes of explanation, numerous specific details are set forth to provide a thorough understanding of the disclosed embodiments. It will be apparent, however, that one or more embodiments may be practiced without these specific details.

The numerical ranges or percentage ranges used in the disclosure shall be deemed to cover all possible subranges and all individual values within the ranges.

The “acyl ester” described in the disclosure refers to an ester compound having an acrylic functional group (RCOOR′) obtained by performing an esterification reaction between an acid compound or an acyl chloride compound and an alcohol compound.

The following will describe in detail, with reference to the drawings, the method of synthesizing acyl ester using a microreactor according to the disclosure.

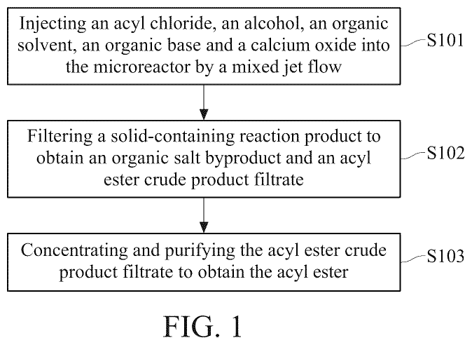

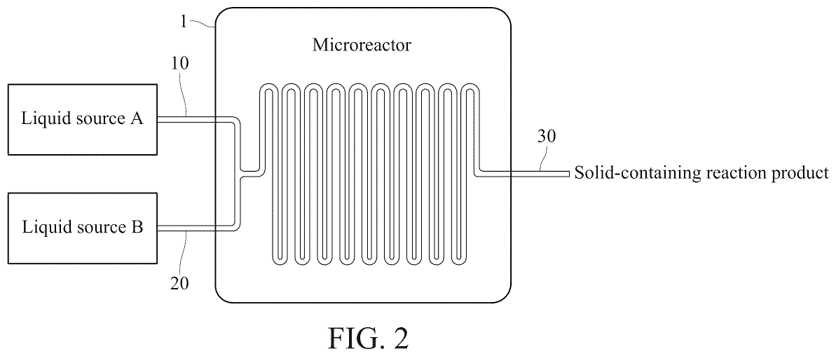

FIG. 1 is a flowchart of the method of synthesizing acyl ester using a microreactor according to one embodiment of the disclosure. FIG. 2 is a schematic diagram of the method of synthesizing acyl ester using the microreactor according to one embodiment of the disclosure.

As shown in FIG. 1, in one embodiment of the disclosure, the method of synthesizing acyl ester using a microreactor includes: injecting an acyl chloride, an alcohol, an organic solvent, an organic base and a calcium oxide into the microreactor by a mixed jet flow (Step S101); filtering a solid-containing reaction product to obtain an organic salt byproduct and an acyl ester crude product filtrate (Step S102); and concentrating and purifying the acyl ester crude product filtrate to obtain the acyl ester (Step S103).

At the start of the reaction, by using a first channel and a second channel, the acyl chloride, the alcohol, the organic solvent, the organic base, and the calcium oxide are injected into the microreactor by the mixed jet flow (Step S101).

In detail, the acyl chloride is injected into the microreactor through the first channel; the calcium oxide is injected into the microreactor through the second channel; and each of the alcohol, the organic base and the organic solvent is independently injected into the microreactor through the second channel of the microreactor after being mixed with the calcium oxide, or injected into the microreactor through the first channel of the microreactor after being mixed with the acyl chloride. As shown in FIG. 2, the components from a liquid source A injected through a first channel 10 and the components from a liquid source B injected through a second channel 20 are mixed and reacted in a microreactor 1 to obtain a solid-containing reaction product, and ultimately, the solid-containing reaction product, which contains an insoluble solid (for example, an organic salt byproduct), is discharged from an outlet 30 of the microreactor 1. The solid-containing reaction product includes the organic salt byproduct and the acyl ester crude product filtrate.

In some embodiments, the acyl chloride may either be injected or the acyl chloride may be dissolved in the organic solvent before being injected into the microreactor through the first channel, and a suspension obtained by mixing the organic solvent, the alcohol, the organic base, and the calcium oxide may be injected into the microreactor through the second channel.

In another embodiments, the acyl chloride may either be injected and mixed with the organic base, or the acyl chloride may be dissolved in the organic solvent before being injected and mixed with the organic base, or the mixture of the acyl chloride and the organic base may be injected into the microreactor through the first channel, and the suspension obtained by mixing the organic solvent, the alcohol, and the calcium oxide may be injected into the microreactor through the second channel.

In yet another embodiments, the acyl chloride may either be injected and mixed with the alcohol, or the acyl chloride may be dissolved in the organic solvent before being injected and mixed with the alcohol, or the mixture of the acyl chloride and the alcohol may be injected into the microreactor through the first channel, and the suspension obtained by mixing the organic solvent, the organic base, and the calcium oxide may be injected into the microreactor through the second channel.

In some embodiments, the organic solvent may include dichloromethane, chloroform, tetrahydrofuran, or a combination thereof. In some embodiments, the organic base may include triethylamine, tributylamine, pyridine, or a combination thereof. In some embodiments, the acyl chloride may include acryloyl chloride, methacryloyl chloride, or a combination thereof. In some embodiments, the alcohol may include 1-methyl-1-cyclohexanolisosorbide, 4,4′-dihydroxybiphenyl, bisphenol A, tris(hydroxymethyl)nitromethane, 4′-hydroxy-4-biphenylcarbonitrile, 4′-((6-hydroxyhexyl)oxy)-[1,1′-biphenyl]-4-carbonitrile, or a combination thereof.

In some embodiments, the acyl ester may include 1-methylcylohexantyl-2-methacrylate, isosorbide diacrylate, biphenyl-4,4′-diyl bis(2-methylacrylate), bisphenol A diacrylate, tris(hydroxymethyl)nitromethane triacrylate, acrylic acid 2-(4′-cyano-biphenyl-4-yloxy)-ethyl ester, 4-[(6-acryloyloxy) hexyloxy]-4′-cyanobiphenyl, or a combination thereof. In some embodiments, the organic salt byproduct may include triethylamine hydrochloride, tributylamine hydrochloride, pyridine hydrochloride, or a combination thereof.

In some embodiments, a molar ratio of the calcium oxide to the alcohol is 1:5 to 1:20. In another embodiments, the molar ratio of the calcium oxide to the alcohol is 1:5 to 1:10. When the acyl chloride reacts with the alcohol in the presence of the organic base, an ester and hydrochloric acid are generated, and the organic base (such as triethylamine) reacts with the hydrochloric acid to form a large-particle size solid byproduct (the organic salt byproduct). In addition, the ester and hydrochloric acid generated by the reaction and the organic base may undergo a hydrochloric acid addition reaction (a side reaction) to form a hydrochloric acid addition byproduct, thereby reducing the reaction yield. The addition of the calcium oxide may allow the hydrochloric acid generated by the reaction to initially react with the calcium oxide, forming small-size calcium chloride particles that suppress the formation of the hydrochloric acid addition byproduct. This helps preventing pipeline clogging and improves reaction yield. Additionally, the combination of the calcium oxide addition and the usage of the microreactor of the disclosure may more effectively reduce the particle size (to less than or equal to 20 μm). In some embodiments, when the molar ratio of the calcium oxide to the alcohol is 1:10, the combination with the usage of the microreactor of the disclosure can reduce the particle size to less than 3 μm.

In some embodiments, a jet flow velocity ratio of the first channel to the second channel may be 6 to 60. In another embodiments, the jet flow velocity ratio of the first channel to the second channel may be 6 to 10. The jet flow velocity ratio=(flow rate of the first channel/flow rate of the second channel)×[(diameter of the second channel)²/(diameter of the first channel)²].

By increasing the jet flow velocity ratio of the first channel and the second channel, the flowability of the solution may be increased, thereby effectively reducing pipeline clogging by the organic salt byproduct. When the jet flow velocity ratio of the first channel to the second channel is below 6, the mixing efficiency of the reactants injected into the microreactor is poor, and the organic salt byproduct is susceptible to agglomeration, leading to an increase in particle size.

Different diameter ratios between the first channel and the second channel will also affect the operating pressure during the reaction. In some embodiments, the diameter ratio of the first channel to the second channel may be 1:2 to 1:5.5. In other embodiments, the diameter ratio of the first channel to the second channel may be 1:4 to 1:5.5. When the diameters of the channels are identical, pipeline clogging occurs, reducing processing stability.

In some embodiments, a reaction time of the above components in the microreactor may be 0.1 seconds to 300 seconds, and a reaction temperature in the microreactor may be 0° C. to 30° C. In another embodiments, the reaction time of the above components in the microreactor may be less than 0.1 seconds. In yet another embodiments, the reaction time of the above components in the microreactor may be less than 0.01 seconds.

Next, the solid-containing reaction product is filtered to obtain the organic salt byproduct and the acyl ester crude product filtrate (Step S102).

In detail, the organic salt byproduct solid contained in the solid-containing reaction product may be separated from the target acyl ester product by using vacuum filtration or other methods capable of separating the solid phase from the liquid phase in the mixture.

Finally, the acyl ester crude product filtrate is concentrated and purified to obtain the acyl ester (Step S103).

In detail, the purification method may include recrystallization, chromatography, or a combination thereof. Through purification, the organic solvent, water generated by the reaction, and/or unreacted reactants in the acyl ester crude product are removed to obtain the target acyl ester product with higher purity. The concentration method may include an atmospheric distillation, a reduced pressure distillation, or a combination thereof.

In some embodiments of the disclosure, the method of synthesizing acyl ester using the microreactor includes: injecting the acyl chloride, the alcohol, the organic solvent, the organic base, and the calcium oxide into the microreactor by the mixed jet flow to react and obtain the acyl ester; wherein the acyl chloride is injected into the microreactor through the first channel; the calcium oxide is injected into the microreactor through the second channel; and each of the alcohol, the organic base, and the organic solvent is independently injected into the microreactor through the second channel of the microreactor after being mixed with the calcium oxide, or injected into the microreactor through the first channel of the microreactor after being mixed with the acyl chloride. The difference between these embodiments and the embodiments of those of FIG. 1 and FIG. 2 are that in these embodiments, after obtaining the acyl ester, Step S102 and Step S103 are not performed. For a detailed description of these embodiments, please refer to Step S101 of the embodiments described with reference to FIG. 1 and FIG. 2.

The method of synthesizing acyl ester using the microreactor of the disclosure reduces the generation of the large-particle size solid organic salt byproduct by adding the calcium oxide, thereby preventing pipeline clogging, and suppressing the generation of the hydrochloric acid addition byproduct to improve the reaction yield. The method of synthesizing acyl ester using the microreactor of the disclosure is performed in the microreactor at room temperature, thereby effectively reducing the energy consumption. Moreover, conducting the synthesis reaction in the microreactor enables continuous production, enhancing processing efficiency. In addition, the method of synthesizing acyl ester using the microreactor of the disclosure only requires filtration after the reaction to obtain the target acyl ester product, simplifying the process and reducing wastewater.

The following will describe in detail the examples and comparative examples of the disclosure.

### Experiment 1: The Effect of Organic Base on the Efficiency of Acyl Ester Synthesis

Jet flow synthesis: 18.1 g (0.2 mol) of propene acyl chloride was injected into a microreactor through a first channel, and 14.6 g (0.1 mol) of isosorbide, 20.2 g (0.2 mol) of triethylamine, 0.56 g (0.01 mol) of the calcium oxide, and 80 g (0.94 mol) of dichloromethane were injected into the microreactor through a second channel, so that the above components were mixed and reacted to obtain an acyl ester product (isosorbide diacrylate). (The diameter ratio of the first channel to the second channel was 1:5.5, and the jet flow velocity ratio of the first channel to the second channel was 6.)

Batch synthesis: 14.6 g (0.1 mol) of isosorbide, 11.2 g (0.2 mol) of the calcium oxide, and 80 g (0.94 mol) of dichloromethane were added to a reaction vessel and mixed by stirring; then, 18.1 g (0.2 mol) of propene acyl chloride was slowly added dropwise into the reaction vessel while stirring thoroughly, allowing the reaction to proceed to obtain the acyl ester product (isosorbide diacrylate).

TABLE 1

| | | Reaction | | Calcium | | Hydrochloric Acid | |
|---|---|---|---|---|---|---|---|
| | Synthetic | Temperature | Organic | Oxide:Alcohol | Reaction | Addition Byproduct | Yield |
| | Mean | (° C.) | Base | (molar) | Time | (%) | (%) |
| Example 1 | Jet flow | 25 | Triethyl- | 1:10 | 5 | 5 | 92 |
| | | | amine | | minutes | | |
| Comparative | Batch | 25 | X | 2:1 | 600 | 7 | 67 |
| Example 1 | | | | | minutes | | |

According to the results of Table 1, in Example 1 with the addition of triethylamine, the acyl ester synthesis reaction could be completed in only 5 minutes with a reaction yield of 92%, and the addition of only 0.1 equivalent of the calcium oxide might be sufficient to achieve the effect of reducing the hydrochloric acid addition byproducts. In contrast, in Comparative Example 1 the absence of trimethylamine), not only exhibited a slower reaction (longer reaction time) but also yielded only 67% of acyl ester. Moreover, Comparative Example 1 produced a large amount of solid precipitate that was non-flowable after the reaction, making it unsuitable for continuous processing. In Example 1, the organic base and 0.1 equivalent of the calcium oxide reacted with the hydrochloric acid produced during the reaction, to form solid particles with smaller particle sizes. On contrary, the product of Comparative Example 1 visibly contained solid particles with larger particle size. The disclosure uses small amount of reagents, requires less reaction time, achieves a high reaction yield, and was suitable for continuous processing system.

### Experiment 2: The Effect of Diameter Ratio on Operating Pressure

18.1 g (0.2 mol) of propene acyl chloride was injected into the microreactor through a first channel (with a diameter “A”), and 14.6 g (0.1 mol) of isosorbide, 20.2 g (0.2 mol) of triethylamine, 0.56 g (0.01 mol) of the calcium oxide, and 80 g (0.94 mol) of dichloromethane were injected into the microreactor through a second channel (with a diameter “B)”, so that the above components were mixed and reacted to obtain the acyl ester product (isosorbide diacrylate). The experimental results were shown in Table 2. (The jet flow velocity ratio of the first channel to the second channel was 6.)

TABLE 2

| | | | Diameter Ratio | Operating |
|---|---|---|---|---|
| | Diameter | Diameter | (Diameter B/ | Pressure |
| | A (mm) | B (mm) | Diameter A) | (atm) |
| Example 2 | 0.8 | 4.4 | 5.5 | ~3 |
| Example 3 | 1.6 | 4.4 | 2.75 | 10 |
| Example 4 | 0.8 | 1.6 | 2 | 18 |
| Comparative | 1.6 | 1.6 | 1 | >20 (Clogging) |
| Example 2 | | | | |

According to the results of Table 2, the operating pressure decreased with an increase in the diameter ratio of the first channel to the second channel. When the diameter ratio of the first channel to the second channel was greater than or equal to 5.5, the operating pressure was about 3 atmospheres (atm), and the processing stability is high. When the diameters of the first and second channels were identical, the suspension injected into the second channel contained calcium oxide solids and exhibited poor flowability, resulting in insufficient calcium oxide to suppress the generation of large-particle size solid organic salt byproducts, which lead to pipeline clogging.

### Experiment 3: The Effect of Jet Flow Ratio on Operating Pressure and Particle Size of Organic Salt Byproduct

18.1 g (0.2 mol) of acrylic acyl chloride was injected into a microreactor through a first channel (with diameter “A” and flow rate “A”), and 14.6 g (0.1 mol) of isosorbide, 20.2 g (0.2 mol) of triethylamine, 0.56 g (0.01 mol) of calcium oxide, and 80 g (0.94 mol) of dichloromethane were injected into the microreactor through a second channel (with diameter B and flow rate B), so that the above components were mixed and reacted to obtain an acyl ester product (isosorbide diacrylate). The experimental results were shown in Table 3. (The diameter ratio of the first channel to the second channel was 1:5.5.)

TABLE 3

| | Flow | Flow | Injecting | | Particle |
|---|---|---|---|---|---|
| | Rate A | Rate B | Velocity | Operating | Size |
| | (mL/min) | (mL/min) | Ratio | Pressure | (μm) |
| Example 5 | 1 | 5 | 6 | 3 | 0.4~3 |
| Example 6 | 5 | 5 | 30 | 4 | 0.4~3 |
| Example 7 | 10 | 5 | 60 | 6 | 0.4~2 |
| Comparative | 1 | 10 | 3 | 11 | 10~50 |
| Example 3 | | | | | |

$${* Jet flow velocity ratio = ( flow rate of the first channel / flow rate of the second channel ) \times [ {( diameter of the second channel )}^{2} / ( diameter of the first channel )}^{2} ]$$

According to the results of Table 3, when the jet flow velocity ratio of the first channel to second channel was too low, the particle size of the organic salt byproduct was larger and the operating pressure was higher. This result was presumed to be due to poor mixing efficiency at low jet flow velocity ratio of the first channel to second channel, which causes the organic salt byproduct to agglomerate and increases its particle size. The increased in solid particle size increased the flow resistance of the fluid, thereby increasing the operating pressure. When the jet velocity ratio of the first channel to second channel was greater than or equal to 6, the agglomeration of the organic salt byproduct couldn be effectively reduced, allowing the microreactor to maintain a stable and low operating pressure.

### Experiment 4: Integrated Comparison (the Effect of Synthesis Method, Temperature, and Calcium Oxide Addition)

Jet flow synthesis: 18.1 g (0.2 mol) of propene acyl chloride was injected into the microreactor through a first channel, and 14.6 g (0.1 mol) of isosorbide, 20.2 g (0.2 mol) of triethylamine, 0.28 g (0.005 mol), 0.56 g (0.01 mol), or 1.12 g (0.02 mol) of calcium oxide, and 80 g (0.94 mol) of dichloromethane were injected into the microreactor through the second channel, so that the above components were mixed and reacted to obtain a acyl ester product (isosorbide diacrylate). (The diameter ratio of the first channel to the second channel was 1:5.5, and the jet flow velocity ratio of the first channel to the second channel was 6.)

Batch synthesis: 14.6 g (0.1 mol) of isosorbide, 20.2 g (0.2 mol) of triethylamine, and 80 g (0.94 mol) of dichloromethane were added into the reaction vessel and mixed by stirring; then, 18.1 g (0.2 mol) of propene acyl chloride was slowly added dropwise into the reaction vessel and stirred thoroughly, allowing the reaction to proceed to obtain a acyl ester product (isosorbide diacrylate). In some experiments, 0.56 g (0.01 mol) or 11.2 g (0.2 mol) of calcium oxide was added.

The synthesis efficiencies of the various experimental groups were shown in Table 4 below.

TABLE 4

| | | Reaction | Calcium | Hydrochloric | | Particle |
|---|---|---|---|---|---|---|
| | Synthetic | temperature | Oxide:Alcohol | Acid Addition | Yield | Size |
| | method | (° C.) | (molar) | Byproduct (%) | (g/h) | (μm) |
| Example 8 | Jet flow | 25 | 1:20 | 6 | 74 | 5~20 |
| Example 9 | Jet flow | 25 | 1:10 | 5 | 77 | 0.4~3 |
| Example 10 | Jet flow | 25 | 1:5 | 5 | 77 | 0.4~3 |
| Example 11 | Jet flow | 0 | 1:10 | 5 | 77 | 3~10 |
| Example 12 | Jet flow | 10 | 1:10 | 5 | 77 | 0.5~10 |
| Comparative | Jet flow | 25 | — | 8 | 71 | 10~180 |
| Example 4 | | | | | | |
| Comparative | Common | 25 | 1:10 | Clogging | Clogging | Unable to |
| Example 5 | microreactor | | | | | analyze |
| | process | | | | | |
| Comparative | Batch | 0 | — | 8 | 9.1 | >200 |
| Example 6 | | | | | | |
| Comparative | Batch | 0 | 1:10 | 5 | 9.4 | 20~200 |
| Example 7 | | | | | | |
| Comparative | Batch | 25 | 1:10 | 15 | 8.3 | 20~200 |
| Example 8 | | | | | | |
| Comparative | Batch | 25 | 2:1 | 7 | ~55 | Large amount |
| Example 9 | | | | | | of |
| | | | | | | precipitation |

*Common reactor process refers to the reactor design disclosed in U.S. Pat. No. 10,537,869 B1.

*The product from Comparative Example 8 was distinctly yellow, while the rest were white and nearly transparent.

According to Table 4, by comparing Example 8 to Example 10 and Comparative Example 4, it might be seen that the addition of the calcium oxide might reduce the formation of the hydrochloric acid addition byproducts and decreased the generation of the large-particle size solid organic salt byproducts. By comparing Example 9, Example 11 and Comparative Example 6 to Comparative Example 8, it might be seen that the method of synthesizing acyl ester using the microreactor of the disclosure, which employed jet flow injection and the microreactor for ester synthesis, achieved a significantly higher reaction yield compared to traditional batch synthesis.

By comparing Example 9 and Example 11, it could be seen that the method of synthesizing acyl ester using the microreactor of the disclosure might also achieve the same reaction yield at room temperature as at 0° C. Therefore, there was no need to use cooling equipment to maintain the system temperature below 0° C., resulting in reduced energy consumption.

By comparing Example 9 and Comparative Example 5, it could be seen that even when acyl ester synthesis was conducted using both a jet flow and a microreactor setup, Comparative Example 5, which was not designed according to the disclosure, exhibited pipeline clogging.

By comparing Example 9 and Comparative Example 9, it could be seen that, despite the attempt to reduce the particle size of the organic salt byproduct by adding a large amount of the calcium oxide, Comparative Example 9 still resulted in a significant amount of precipitate when the jet flow and microreactor were not used. Moreover, the product of Comparative Example 7 (which contained both liquid and solid) exhibited a distinctly yellow color, indicating a yellowing phenomenon that was undesirable for industrial applications.

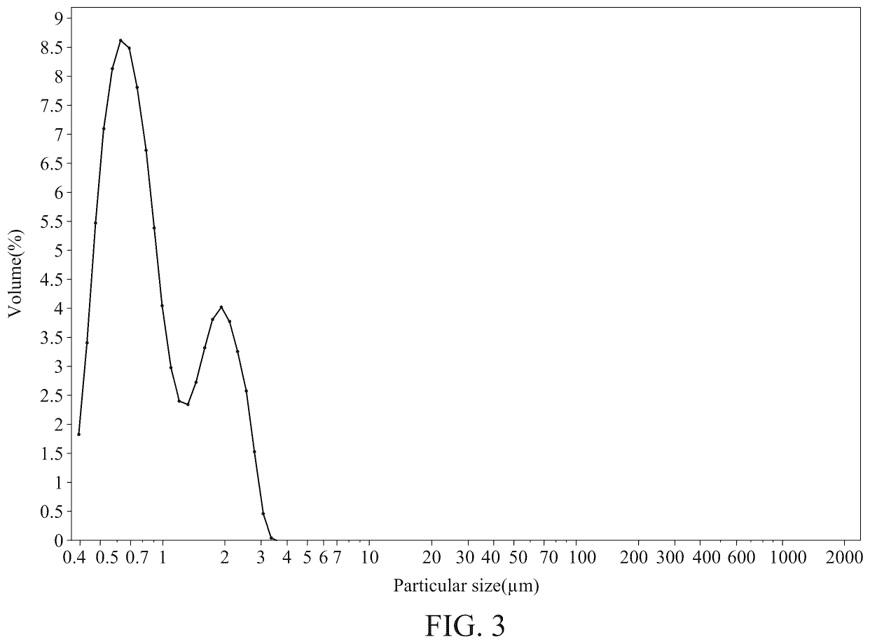

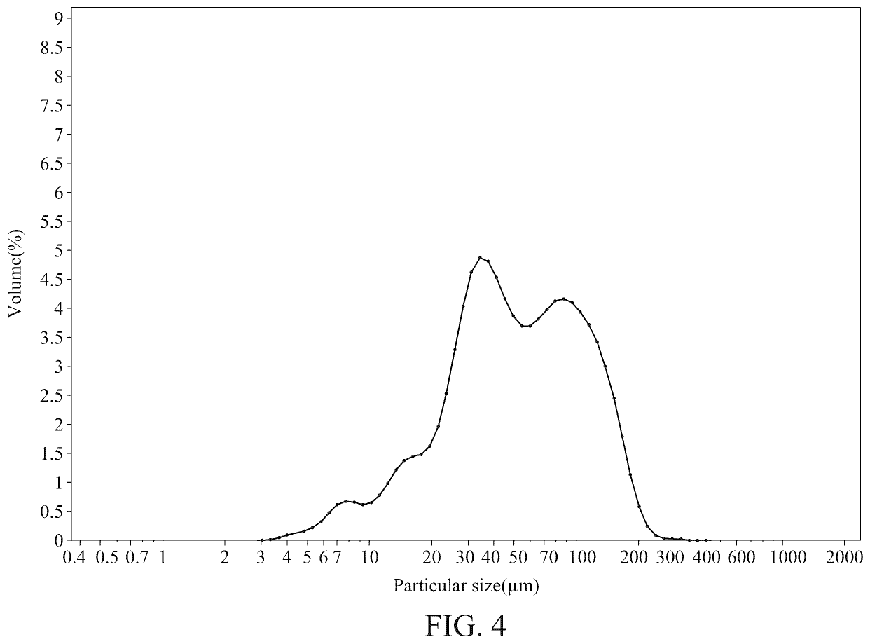

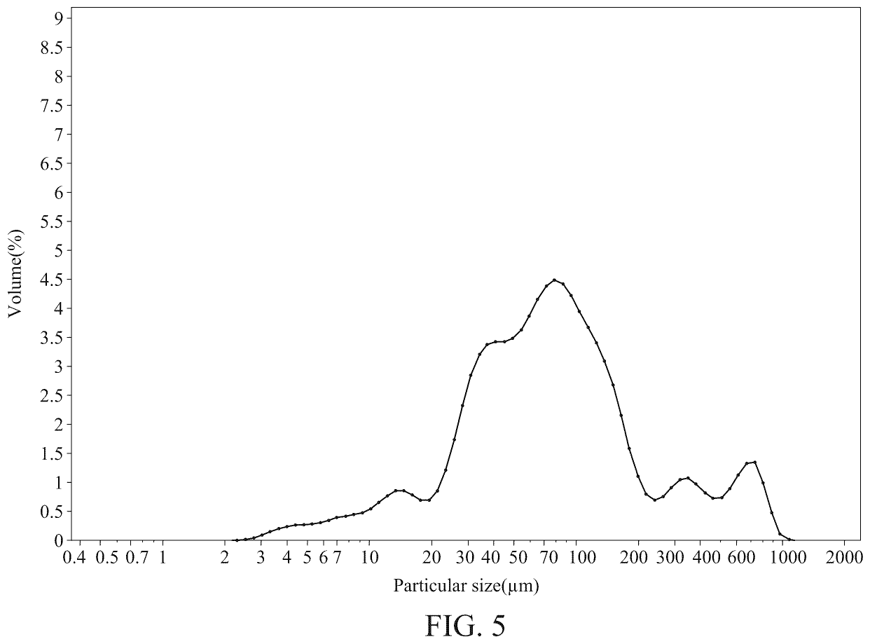

FIG. 3 was a particle size diagram of the solid organic salt byproducts generated during the synthesis of acyl ester using the microreactor according to Example 9; FIG. 4 was a particle size diagram of the solid organic salt byproducts generated during the synthesis of acyl ester using the microreactor according to Comparative Example 4; and FIG. 5 was a particle size diagram of the solid organic salt byproducts generated during the synthesis of acyl ester using the microreactor according to Comparative Example 8.

According to FIG. 3 to FIG. 5, it could be seen that by adding the calcium oxide and using the microreactor to maintain continuous flow during the acyl ester synthesis, the agglomeration of the organic salt byproduct was effectively reduced, thereby decreasing the particle size of the organic salt byproduct.

### Experiment 5: Synthesis of Other Acyl Esters

The acyl chloride and the alcohol were synthesized by jet flow as shown in Table 5 below: 0.2 mol of acyl chloride was injected into the microreactor through a first channel, and 0.1 mol of alcohol, 20.2 g (0.2 mol) of triethylamine, 0.56 g (0.01 mol) of the calcium oxide, and 80 g (0.94 mol) of dichloromethane were injected into a microreactor through a second channel, so that the above components were mixed and reacted to obtain the acyl ester product. Among them, the acyl ester products in Example 11 to Example 17 and Comparative Example 12 to Comparative Example 18 were, respectively, 1-methylcylohexantyl-2-methacrylate, 4-[(6-acryloyloxy) hexyloxy]-4′-cyanobiphenyl, acrylic acid 2-(4′-cyano-biphenyl-4-yloxy)-ethyl ester, isosorbide diacrylate, biphenyl-4,4′-diyl bis(2-methylacrylate), bisphenol A diacrylate, and tris(hydroxymethyl)nitromethane triacrylate. (The diameter ratio of the first channel to the second channel was 1:5.5, the injecting velocity ratio of the first channel to the second channel was 6, and the reaction time was 5 minutes.)

TABLE 5

| | | | | | Hydrochloric | |
|---|---|---|---|---|---|---|
| | | | Reaction | Calcium | Acid Addition | |
| | | | Temperature | Oxide:Alcohol | Byproduct | |
| | Acyl chloride | Alcohol | (° C.) | (molar) | (%) | Yield (%) |
| Example 11 | Methacryloyl | 1-methyl-1- | 25 | 1:10 | 8 | 72 |
| | chloride | cyclohexanol | | | | |
| Example 12 | Acryloyl | 4′-((6- | 25 | 1:10 | 7 | 85 |
| | chloride | hydroxyhexyl)oxy)-[1,1′- | | | | |
| | | biphenyl]-4- | | | | |
| | | carbonitrile | | | | |
| Example 13 | Acryloyl | 4′-hydroxy-4- | 25 | 1:10 | <1 | 95 |
| | chloride | biphenylcarbonitrile | | | | |
| Example 14 | acryloyl | Isosorbide | 25 | 1:10 | 5 | 92 |
| | chloride | | | | | |
| Example 15 | Methacryloyl | 4,4′- | 25 | 1:10 | 10 | 69 |
| | chloride | dihydroxybiphenyl | | | | |
| Example 16 | Acryloyl | Bisphenol A | 25 | 1:10 | 8 | 80 |
| | chloride | | | | | |
| Example 17 | Acryloyl | Tris(hydroxymethyl)nitromethane | 25 | 1:10 | 3 | 85 |
| | chloride | | | | | |
| Comparative | Methacryloyl | 1-methyl-1- | 25 | — | 10 | 67 |
| Example 12 | chloride | cyclohexanol | | | | |
| Comparative | Acryloyl | 4′-((6- | 25 | — | 12 | 75 |
| Example 13 | chloride | hydroxyhexyl)oxy)-[1,1′- | | | | |
| | | bipheny1]-4- | | | | |
| | | carbonitrile | | | | |
| Comparative | Acryloyl | 4′-hydroxy-4- | 25 | — | 2 | 90 |
| Example 14 | chloride | biphenylcarbonitrile | | | | |
| Comparative | Acryloyl | Isosorbide | 25 | — | 8 | 69 |
| Example 15 | chloride | | | | | |
| Comparative | Methacryloyl | 4,4′- | 25 | — | 12 | 45 |
| Example 16 | chloride | dihydroxybiphenyl | | | | |
| Comparative | Acryloyl | Bisphenol A | 25 | — | 12 | 73 |
| Example 17 | chloride | | | | | |
| Comparative | Acryloyl | Tris(hydroxymethyl)nitromethane | 25 | — | 7 | 78 |
| Example 18 | chloride | | | | | |

According to the results of Table 5, the method of synthesizing acyl esters using a microreactor of the disclosure might be used to synthesize acyl esters of different chemical structures. In addition, by adding the calcium oxide, the formation of hydrochloric acid addition byproducts might be reduced and the reaction yield might be improved.

It will be apparent to those skilled in the art that various modifications and variations can be made to the disclosed Embodiments. It is intended that the specification and examples be considered as exemplary only, with the true scope of the disclosure being indicated by the following claims and their equivalents.

What is claimed is:

1. A method of synthesizing acyl ester using a microreactor, comprising:
injecting an acyl chloride, an alcohol, an organic solvent, an organic base and a calcium oxide into a microreactor by a mixed jet flow,
wherein the acyl chloride is injected into the microreactor through a first channel of the microreactor;
wherein the calcium oxide is injected into the microreactor through a second channel of the microreactor; and
wherein each of the alcohol, the organic base and the organic solvent is independently injected into the microreactor through the second channel of the microreactor after being mixed with the calcium oxide, or injected into the microreactor through the first channel of the microreactor after being mixed with the acyl chloride, and a jet flow velocity ratio of the first channel to the second channel is 6 to 60.

2. The method as claimed in claim 1, wherein the organic solvent comprises dichloromethane, chloroform, tetrahydrofuran, or a combination thereof.

3. The method as claimed in claim 1, wherein the organic base comprises triethylamine, tributylamine, pyridine, or a combination thereof.

4. The method as claimed in 1, wherein the acyl chloride comprises acryloyl chloride, methacryloyl chloride, or a combination thereof.

5. The method as claimed in claim 1, wherein the alcohol comprises 1-methyl-1-cyclohexanol, isosorbide, 4,4′-dihydroxybiphenyl, bisphenol A, tris(hydroxymethyl)nitromethane, 4′-hydroxy-4-biphenylcarbonitrile, 4′-((6-hydroxyhexyl)oxy)-[1,1′-biphenyl]-4-carbonitrile, or a combination thereof.

6. The method as claimed in claim 1, wherein the acyl ester comprises 1-methylcylohexantyl-2-methacrylate, isosorbide diacrylate, biphenyl-4,4′-diyl bis(2-methylacrylate), bisphenol A diacrylate, tris(hydroxymethyl)nitromethane triacrylate, acrylic acid 2-(4′-cyano-biphenyl-4-yloxy)-ethyl ester, 4-[(6-acryloyloxy) hexyloxy]-4′-cyanobiphenyl, or a combination thereof.

7. The method as claimed in claim 1, wherein the organic salt byproduct of the method comprises triethylamine hydrochloride, tributylamine hydrochloride, pyridine hydrochloride, or a combination thereof.

8. The method as claimed in claim 1, wherein
the acyl chloride dissolved in the organic solvent or the acyl chloride is injected into the microreactor through the first channel; and
the organic solvent, the alcohol, the organic base and the calcium oxide are injected into the microreactor through the second channel.

9. The method as claimed in claim 1, wherein
the acyl chloride or the acyl chloride dissolved in the organic solvent and the organic base are injected into the microreactor through the first channel; and
the organic solvent, the alcohol and the calcium oxide are injected into the microreactor through the second channel.

10. The method as claimed in claim 1, wherein
the acyl chloride or the acyl chloride dissolved in the organic solvent and the alcohol are injected into the microreactor through the first channel; and
the organic solvent, the organic base and the calcium oxide are injected into the microreactor through the second channel.

11. The method as claimed in claim 1, wherein a diameter ratio of the first channel to the second channel is 1:2 to 1:5.5.

12. The method as claimed in claim 1, wherein a reaction temperature in the microreactor is 0° C. to 30° C.

13. The method as claimed in claim 1, wherein a molar ratio of the calcium oxide to the alcohol is 1:5 to 1:20.
